(19) United States
(12) Patent Application Publication
(10) Pub. No.: US 2004/0116385 A1
(43) Pub. Date: Jun. 17, 2004

(54) Treating and preventing viral infections with porphyrin-based compounds

(76) Inventors: Maria da Graca Henriques Vicente, Baton Rouge, LA (US); Luigi G. Marzilli, Baton Rouge, LA (US)

Correspondence Address: PATENT DEPARTMENT, TAYLOR, PORTER, BROOKS & PHILLIPS, L.L.P, P.O. BOX 2471, BATON ROUGE, LA, 70821-2471, US

(21) Appl. No.: 10/712,228

(22) Filed: Nov. 12, 2003

Related U.S. Application Data
(60) Provisional application No. 60/426,062, filed on Nov. 13, 2002.

(51) Int. Cl. A61K 31/69

(52) U.S. Cl. 514/64

(57) ABSTRACT

Porphyrins containing one or more negatively-charged, amphiphilic nido-carborane substituents have antiviral or virucidal activity. The most active compounds tested to date are negatively charged, amphiphilic, and water-soluble. The negative charges lie primarily in the boron clusters. The carbon-carbon bonds linking the boron-containing groups to the porphyrin ring make the compounds highly resistant to hydrolysis. These compounds have strong potential for use as antiviral and virucidal drugs, as they are highly stable, water-soluble, negatively-charged, amphiphilic, and have low toxicity to normal mammalian cells. Preliminary tests in vitro have shown high activity against HIV.

The benefit of the filing date of provisional application serial No. 60/426,062, filed Nov. 13, 2002, is claimed under 35 U.S.C. § 119(e).

The development of this invention was partially funded by the Government under grant number P01 Al45883 awarded by the National Institutes of Health. The Government has certain rights in this invention.

This invention pertains to the prevention and treatment of viral infections.

There is an unfilled need for improved antiviral and virucidal agents worldwide. In particular, there is a great need for improved, effective, anti-viral drugs against the human immunodeficiency virus (HIV), which causes acquired immunodeficiency syndrome (AIDS). Porphyrins have been investigated as potential antiviral agents, but none have been approved for this use to date. Two porphyrins, Photofrin™ and Visudyne™, have been approved by the United States Food and Drug Administration for use in photodynamic treatment of certain cancers.

A. Debnath et al., “Anti-HIV-1 activity of carborane derivatives of porphyrins,” Med. Chem. Res., vol. 9, pp. 267-275 (1999) reported that certain boronated porphyrins possessed anti-HIV activity, namely, blocking the gp120 site, and inhibiting infection of a CD4+ T-cell line.

A. Debnath et al., “Three-dimensional structure-activity analysis of a series of porphyrin derivatives with anti-HIV-1 activity targeted to the V3 loop of the gp 120 envelope glycoprotein of the human immunodeficiency virus type 1,” J. Med. Chem., vol. 37, pp. 1099-1108 (1994) discusses certain experimental results and modeling calculations said to support a hypothesis that porphyrins containing anionic and hydrophobic groups might interact with some of the highly conserved positively charged and hydrophobic sites, respectively, of the gp 120 V3 loop of HIV.

D. DeCamp et al., “Specific inhibition of HIV-1 protease by boronated porphyrins,” J. Med. Chem., vol. 35, pp. 3426-3428 (1992) reported that certain boronated porphyrins inhibit HIV-1 protease.

R. Levere et al., “Heme inhibits human immunodeficiency virus 1 replication in cell cultures and enhances the antiviral effect of zidovudine,” Proc. Natl. Acad. Sci. USA, vol. 88, pp. 1756-1759 (1991) reported that heme (which is a metalloporphyrin), either alone or in combination with AZT, could inhibit replication of certain strains of HIV in cells in vitro.

M. Vicente, “Porphyrin-based sensitizers in the detection and treatment of cancer: recent progress,” Curr. Med. Chem., vol. 1, pp. 175-194 (2001) provides a review of the use of porphyrins for cancer detection and treatment by photodynamic therapy, boron neutron capture therapy, radiation therapy, and magnetic resonance imaging.

U.S. Pat. No. 5,109,016 discloses compositions for the inhibition of human immunodeficiency virus containing one or more porphyrin or porphyrin-like compounds; and also discloses assays for testing the efficacy of antiviral compounds.

M. Vicente et al., international patent application WO 01/85736 (2001), discloses the use of the same class of porphyrin-based compounds that are used in the present invention, but for a different purpose, namely, in boron neutron capture therapy for treatment of cancer. See also M. Vicente et al., “Syntheses and preliminary biological studies of four meso-tetra[(nido-carboranylmethyl)phenyl]porphyrins,” Bioorganic & Medicinal Chem., vol. 10, pp. 481-492 (2002); A. Maderna et al., “Synthesis of a porphyrin-labelled carboranyl phosphate diester: a potential new drug for boron neutron capture therapy of cancer,” Chem. Commun., pp. 1784-1785(2002); R. Lauceri etal., “Interactions of anionic carboranylated porphyrins with DNA,” J. Am. Chem. Soc., vol. 123, pp. 5835-5836 (2001); M. Vicente et al., “Syntheses of carbon-carbon linked carboranylated porphyrins for boron neutron capture therapy of cancer,” Tetr. Lett., vol. 41, pp. 7623-7627 (2000); and M. Vicente et al., “Synthesis, dark toxicity and induction of in vitro DNA photodamage by a tetra(4-nido-carboranyl)porphyrin,” J. Photochemistry and Photobiology. Vol. 68, pp. 123-132 (2002).

To the inventors' knowledge, no prior reference has suggested that porphyrins containing nido-carborane cages would be useful as antiviral or virucidal agents.

We have discovered that certain porphyrins containing one or more negatively-charged, amphiphilic nido-carborane substituents are surprisingly effective as antiviral or virucidal agents. (“Antiviral” refers to treatment of an existing viral infection, while “virucidal” refers to prophylaxis to prevent viral infection.)

Preliminary experiments have shown high levels of antiviral activity for these compounds. In particular, high levels of activity against HIV-1 in vitro has been observed for Compounds 16, 31, and 33 as depicted in Schemes 3 and 6. The most. active compounds tested to date are negatively charged, amphiphilic, and water-soluble. A nido-carborane group is depicted as structure 51 in FIG. 7(a).

Examples of active compounds include the compounds depicted as structures 52, 53, and 54 in FIGS. 7(b)-(d). In these structures, M may be a metal ion, preferably a diamagnetic or paramagnetic metal ion such as Zn(II), Cu(II), or Al(III), or it may be 2H, the last resulting in a metal-free macrocycle. Using a diamagnetic metal ion in the compound can assist in the photodynamic treatment against viruses; it can help generate singlet oxygen; and it can assist in strengthening fluorescence, which can make it easier to detect. As just one example of the use of the compounds in photodynamic treatment, the compounds may be used to treat a laboratory coat, laboratory equipment, or medical equipment; exposing the treated object to light of the appropriate wavelength then enhances the compound's antiviral and virucidal properties. Although the preferred diamagnetic metal ions are depicted in structures 52, 53, and 54, more generally, M in these structures may be a metal ion.

The negative charges lie primarily in the boron clusters. The carbon-carbon bonds linking the boron-containing groups to the porphyrin ring make the compounds highly resistant to hydrolysis. By contrast, most priorwork with boron-containing porphyrin compounds as antivirals has relied on ester linkages, which are susceptible to hydrolysis in vivo. Most such prior work has used neutral boron clusters, instead of the negatively-charged boron clusters of the present invention.

These compounds have strong potential for use as antiviral and virucidal drugs, as they are highly stable, water-soluble, negatively-charged, amphiphilic, and have low toxicity to normal mammalian cells.

Preliminary tests in vitro have shown that some of these compounds have high activity against HIV.

The mechanism of action is currently unknown. Without wishing to be bound by this theory, it is possible that these compounds bind to positive charges on the viral envelope, inhibiting the virus' ability to enter or infect a host cell.

### BRIEF DESCRIPTION OF THE DRAWINGS

FIG. 1 (Scheme 1) depicts the synthesis of compounds 1-6.

FIG. 2 (Scheme 2) depicts the synthesis of compounds 7-12.

FIG. 3 (Scheme 3) depicts the synthesis of compounds 13-18.

FIG. 4 (Scheme 4) depicts the synthesis of compounds 19-24.

FIG. 5 (Scheme 5) depicts the synthesis of compounds 25-28.

FIG. 6 (Scheme 6) depicts the synthesis of compounds 27-33.

FIG. 7(a) depicts a nido-carborane group.

FIGS. 7(b)-(d) depict examples of active compounds.

This invention pertains to the antiviral and virucidal use of certain porphyrin compounds containing carboranyl groups attached to the porphyrin group by a carbon-carbon linkage. These porphyrin compounds generally correspond to formula I:

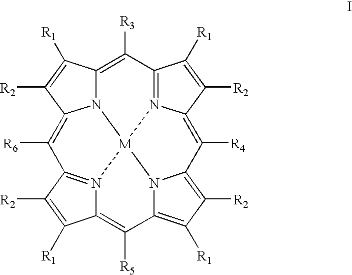

where M is 2H or a metal ion, preferably a diamagnetic or paramagnetic metal ion; R1 and R2 are each independently hydrogen, alkyl or hydroxyalkyl; and R3 through R6 are each independently hydrogen, phenyl, or a substituted phenyl group.

Where one or more of R3 through R6 comprise a substituted phenyl group, that substituted phenyl group corresponds to general formula II:

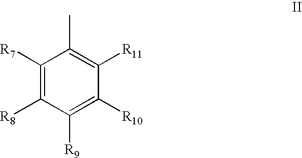

where R7 through R11 are each independently hydrogen, halide, hydroxide, alkoxide, sulfonate,or a substituted or unsubstituted alkyl, aryl or carboranyl group. The carboranyl group should be attached to the phenyl group by a carbon-carbon linkage (and not, for example, an ether linkage or an ester linkage). Typically, one or two of R7 through R11 are nido-carboranyl groups.

At least one of R3 through R6 should be a substituted phenyl group of general formula II, having at least one carboranyl group attached by a carbon-carbon linkage. More preferably, at least two of R3 through R6 should be a substituted phenyl group of general formula II, each having at least one carboranyl group attached by a carbon-carbon linkage. Most preferably, all of R3 through R6 should be a substituted phenyl group of general formula II, each having at least one carboranyl group attached by a carbon-carbon linkage.

In a preferred embodiment, the compound has from 1 to 8 nido-carboranyl substituents.

The carboranyl-containing porphyrin compounds used in this invention may be synthesized, for example, by the following general method. (Examples of specific syntheses are described in detail below.) A pyrrole or dipyrrole is reacted with a benzaldehyde using an acid catalyst, such as trifluoroacetic acid. The pyrrole or dipyrrole may be unsubstituted or substituted, for example with alkyl groups. The benzaldehyde has general formula III:

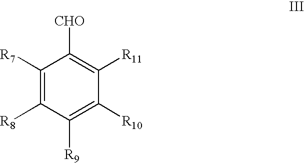

where R7 through R11 are each independently hydrogen, halide, hydroxide, alkoxide, sulfonate,or a substituted or unsubstituted alkyl, aryl or carboranyl group. The carboranyl group should be attached to the phenyl group by a carbon-carbon linkage (and not, for example, an ether linkage or an ester linkage). Typically, one or two of R7 through R 11 are nido-carboranyl groups. Next, the pH of the reaction mixture is lowered until the reaction product is converted to a porphyrin compound. In a preferred version, trifluoroacetic acid is added to the reaction mixture to lower the pH. The next step is oxidation of the reaction mixture, for example, with tetrachloroquinone or dichlorodicyanobenzoquinone.

Optionally, the synthesis may include complexing the porphyrin compound with a metal ion, e.g., by treating the free base of the porphyrin compound with zinc chloride to form a Zn(II) complex.

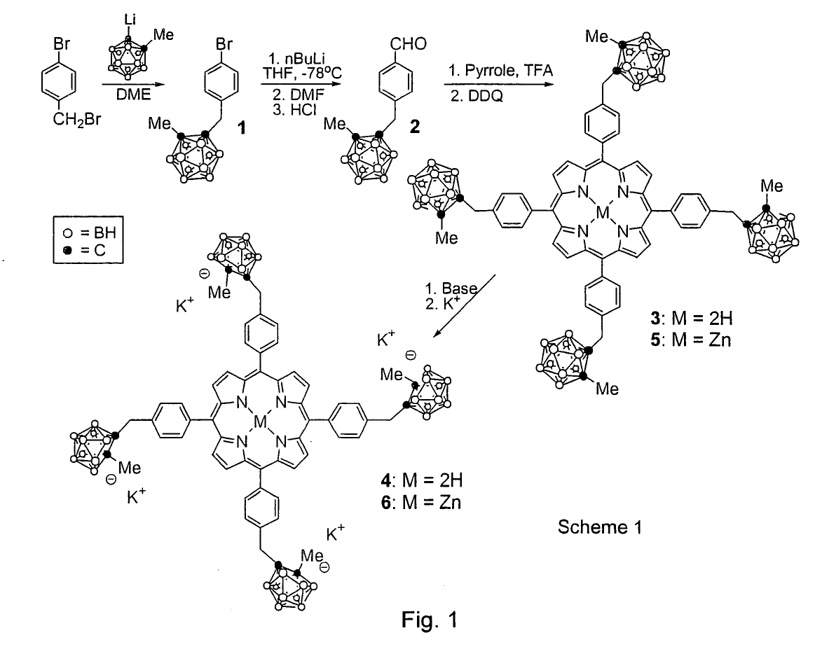

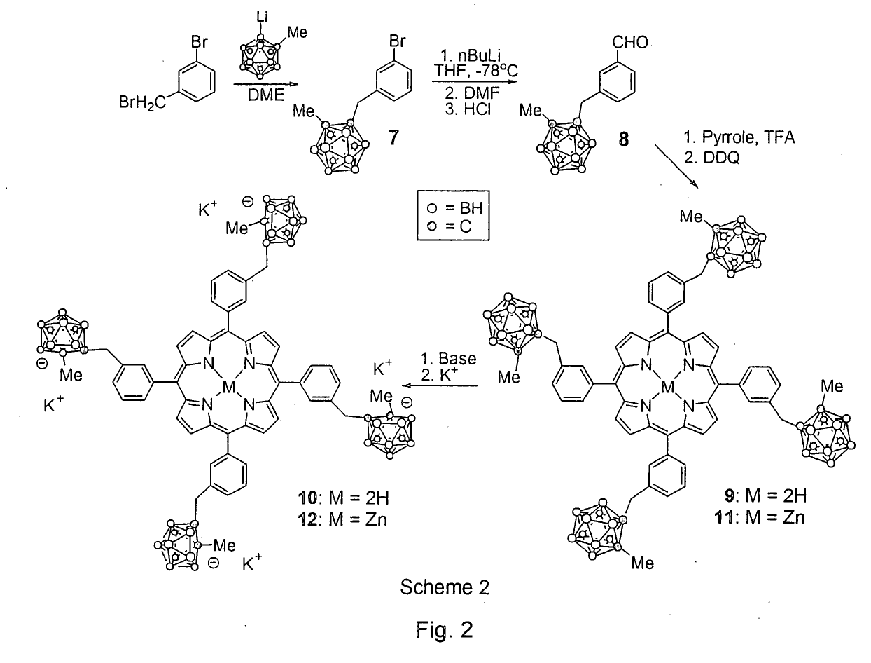

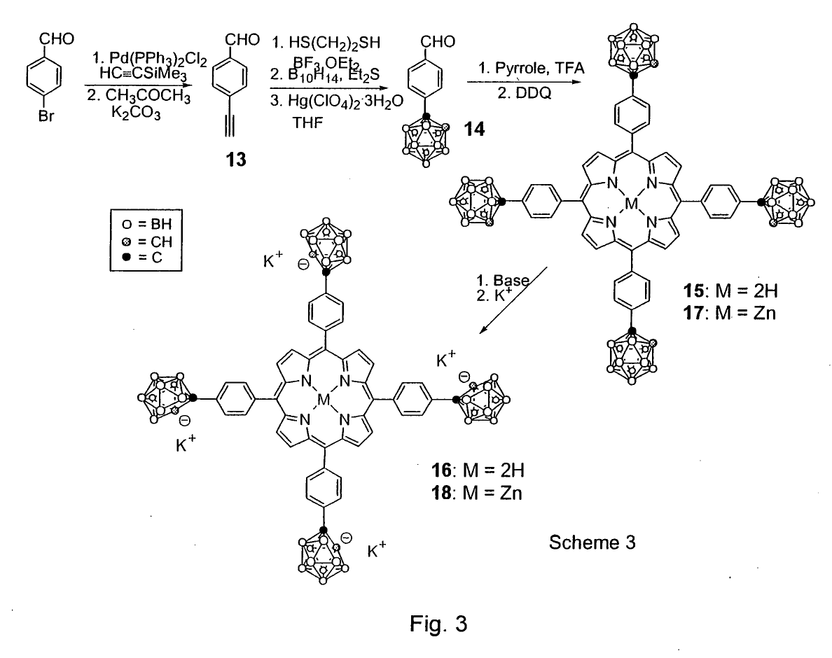

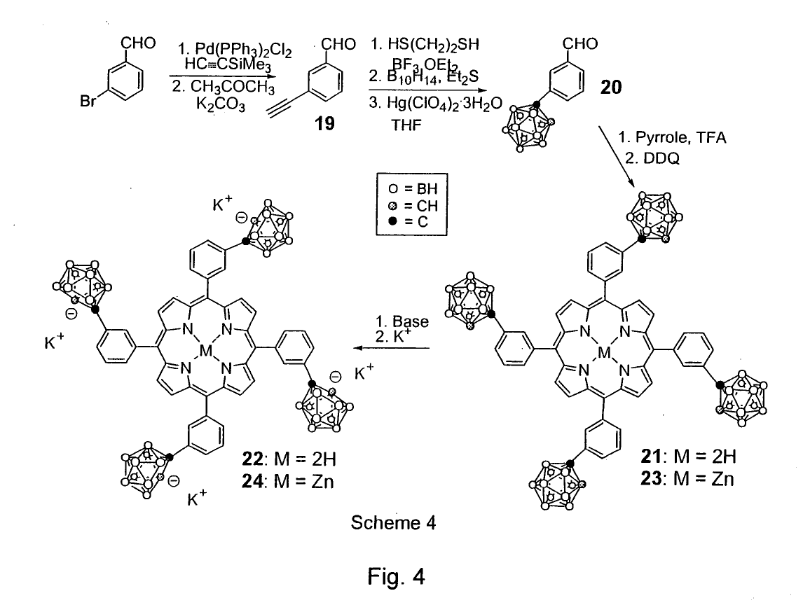

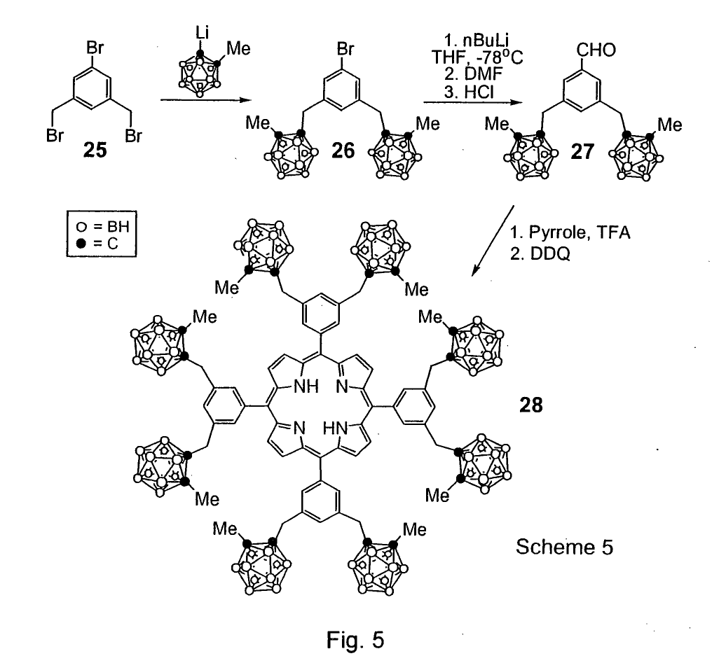

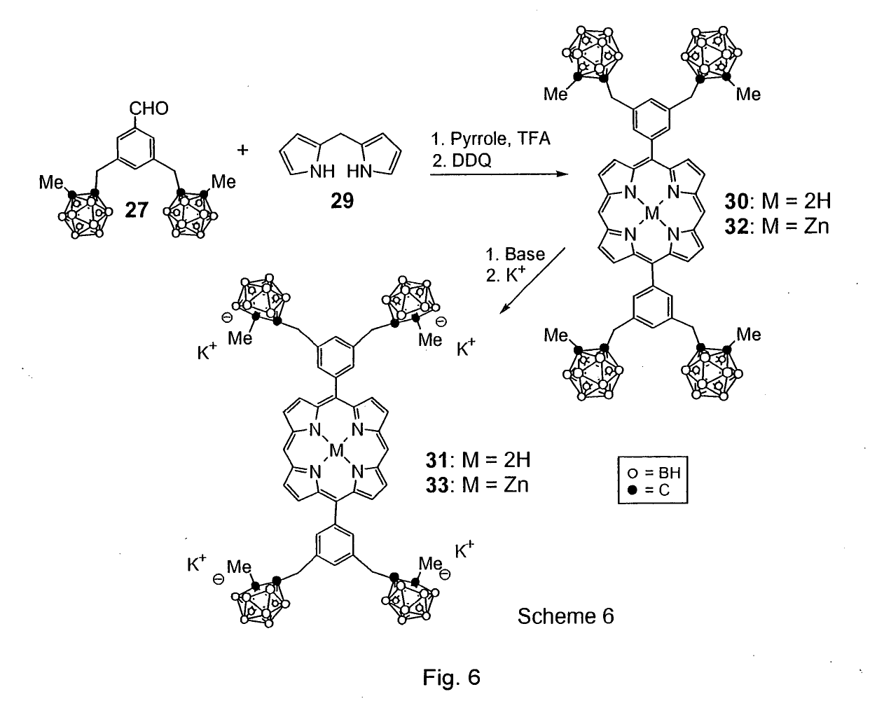

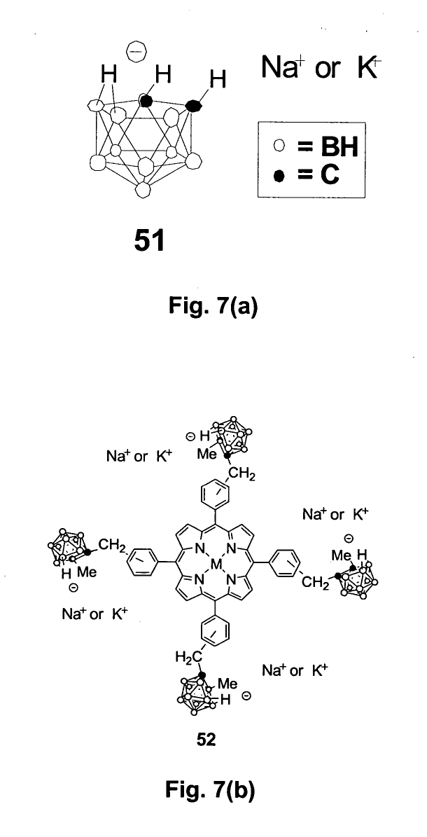

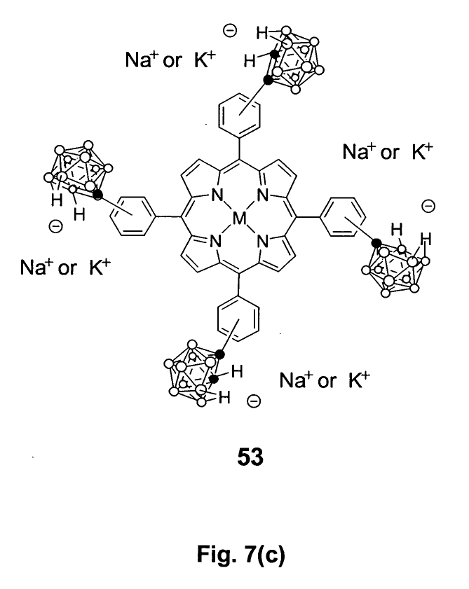

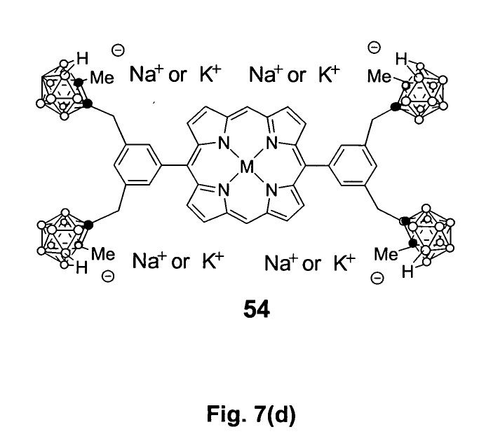

FIGS. 1 through 6 depict, in a few steps, the total synthesis of various carboranylated phenylporphyrins. These meso-phenylporphyrin compounds contain carbon-carbon linkages between the carboranyl groups and the porphyrin ring for increased chemical stability in vitro and in vivo. In addition, the high solubility of these compounds in aqueous solution allows for their easy administration into the blood stream (e.g., via a concentrated saline solution of the drug), without the need for a co-solvent. Preliminary in vitro results show that these new compounds are very promising drugs for both antiviral and virucidal applications. Further in vitro and in vivo studies of these compounds are being conducted.

In summary, the compounds preferred for use in the present invention contain carbon-carbon linkages between a porphyrin ring and carboranyl groups, and have amphiphilic properties both to allow adequate solubility in the blood stream, and to facilitate interaction with viral envelopes.

### EXAMPLES

Synthesis of Compound 1:

[4-(1-methyl-o-carboranyl)methyl]bromobenzene (1): A two-necked round bottom flask containing 1-methyl-o-carborane (5.00 g, 31.65 mmol) in dry DME (150 mL) was cooled to 0° C. under argon. n-BuLi (20.0 mL, 1.6 M in hexane) was added dropwise, and the resulting mixture was stirred at 0° C. for 30 minutes. A solution of 4-(bromomethyl)bromobenzene (7.91 g, 31.65 mmol) in dry DME (15 mL) was added dropwise. After stirring at 0° C. for 10 minutes, the final reaction mixture was warmed to room temperature and then refluxed for 12 hours under argon. The solvent was then removed under vacuum, and the crude solid obtained was purified by recrystallization from dichloromethane/methanol to give the title compound (7.80 g, 75.4% yield) as white crystals. MS (El) m/e 327.1 (M⁺); ¹H-NMR (CDCl₃) δ ppm: 1.3-3.0 (br, 10H, BH), 2.15 (s, 3H, CH₃), 3.41 (s, 2H, CH₂), 7.06 (d, 2H, ArH, J=8.1 Hz), 7.48 (d, 2H, ArH, J=8.1 Hz).

Synthesis of Compound 2:

[4-(1-methyl-o-carboranyl)methyl]benzaldehyde (2): A solution of compound 1 (4.00 g, 12.23 mmol) in THF (150 mL) under argon was cooled to −78° C. (acetone/dry ice bath). n-BuLi (7.6 mL, 1.6 M in hexane) was added dropwise while maintaining the temperature at −78° C. The reaction mixture was stirred for 30 minutes at −78° C., after which dry DMF (5.0 mL, 64.6 mmol) was slowly added. The final mixture was stirred at −78° C. for 15 minutes, and then warmed slowly to room temperature. A 2 N HCl solution (150 mL) was added, and the reaction mixture stirred for 2 h at room temperature. The solution was then reduced to a volume of 200 mL and extracted with dichloromethane (4×50 mL). The organic extracts were washed once with a saturated aqueous NaHCO₃ solution, and once with water, and were then dried over anhydrous Na₂SO₄. After removal of the solvent under vacuum, the oily residue was purified by column chromatography on silica gel (dichloromethane/petroleum ether 1:1), yielding the title compound (2.1 g, 62% yield) as a white solid. MS (El) m/e 276.2 (M⁺); ¹H-NMR (CDCl₃) δ ppm: 1.5-3.0 (br, 10H, BH), 2.19 (s, 3H, CH₃), 3.54 (s, 2H, CH₂), 7.38 (d, 2H, ArH, J=8.0 Hz), 7.89 (d, 2H, ArH, J=8.0 Hz), 10.04 (s, 1H, CHO).

Synthesis of Compound 3:

meso-tetra[4-(1-methyl-o-carboranyl)methylphenyl]porphyrin (3): A solution of aldehyde 2 (1.16 g, 4.19 mmol) and freshly distilled pyrrole (0.30 mL, 4.32 mmol) in dry dichloromethane (420 mL) was purged with argon for 15 minutes. TFA (0.25 mL, 3.15 mmol) was added to the solution, and the final mixture was stirred at room temperature under argon for 20 hours (at which time the starting compound 2 had essentially disappeared completely, as assayed by TLC). After addition of p-chloranil (0.788 g, 3.14 mmol), the final reaction mixture was stirred at room temperature for 2 hours. The solution was concentrated under vacuum to 200 mL, then washed once with a saturated aqueous NaHCO₃ solution, and once with water, and were then dried over anhydrous Na₂SO₄. The residue after removal of the solvent undervacuum was purified by column chromatography (dichloromethane/petroleum ether 1:1), and the fastest-running porphyrin fraction was collected and recrystallized from dichloromethane/methanol, yielding 0.289 g (21% yield) of the title compound as purple crystals, m.p.>300° C.; MS (MALDI) m/e 1296.0 (M⁺); ¹H-NMR (CDCl₃) δ ppm: −2.80 (br, 2H, NH), 1.6-3.1 (br, 40H, BH), 2.34 (s, 12H, CH₃), 3.81 (s, 8H, CH₂), 7.59 (d, 8H, ArH, J=8.0 Hz), 8.20 (d, 8H, ArH, J=8.0 Hz), 8.85 (s, 8H, β-H). UV-Vis (CHCl₃) λₘₐₓ: 418 nm (ε 467,700), 514 (16,867), 550 (8,132), 590 (5,470), 646 (4,028).

Synthesis of Compound 4:

meso-tetra[4-(1-methyl-nido-arboranyl)methylphenyl]porphyrin tetrapotassium salt (4): Porphyrin 3 (0.050 g, 0.0386 mmol) was dissolved in a 3:1 mixture of pyridine and piperidine (4.0 mL), and then stirred at room temperature in the dark for 36 hours under argon. The solvent was completely removed under vacuum, the residue was re-dissolved in a 60% acetone aqueous solution, which was then passed slowly over a Dowex 50W2-100 resin in the potassium form. The porphyrin fraction was collected, dried under vacuum, re-dissolved in a 30% aqueous acetone solution, and again passed through the ion-exchange resin. After removal of the solvent under vacuum, the tetraanionic porphyrin was recrystallized from methanol/diethyl ether, yielding 0.051 g (94% yield) of the title compound, m.p.>300° C. ¹H-NMR (CD₃COCD₃) δ ppm: −2.70 (s, 2H, NH), −2.45 to −1.90 (br, 4H, BH), 0.9-2.4 (br, 32H, BH), 1.59 (s, 12H, CH₃), 3.50 (s, 8H, CH₂), 7.81 (d, 8H, ArH, J=8.0 Hz), 8.08 (d, 8H, ArH, J=8.0 Hz), 8.90 (s, 8H, β-H). UV-Vis (acetone) λₘₐₓ: 420 nm (ε 349,700), 516 (13,595), 554 (12,410), 594 (4,130), 650 (5,990).

Synthesis of Compound 5:

Zn(II)-meso-tetra[4-(1-methyl-o-carborane)methylphenyl]porphyrin (5): To a solution of porphyrin 3 (0.150 g) in dichloromethane (150 mL), THF (10 mL), and pyridine (0.5 mL) were added with ZnCl₂.2H₂O, and the final mixture was stirred at room temperature under argon overnight. The mixture was then washed once with water, dried over anhydrous Na₂SO₄, and the solvent evaporated under vacuum. The residue was purified by column chromatography (dichloromethane/petroleum ether 1:1.5), and the pink-colored fraction was collected and recrystallized from dichloromethane/methanol to give 0.135 g (92% yield) of the title compound as purple crystals, m.p.>300° C.; MS m/e 1358.6; ¹H-NMR (CDCl₃) δ ppm: 1.6-3.0 (br, 40H, BH), 2.33 (s, 12H, CH₃), 3.80 (s, 8H, CH₂), 7.57 (br s, 8H, ArH), 8.19 (br s, 8H, ArH), 8.95 (br s, 8H, β-H). UV-Vis (CHCl₃) λₘₐₓ: 424 nm (ε 577,000), 554 (20,102), 596 (6,380).

Synthesis of Compound 6:

Zn(II)-meso-tetra[4-(1-methyl-nido-carboranyl)methylphenyl]porphyrin tetrapotassium salt (6): The Zn(II) complex 5 (0.075 g, 0.055 mmol) was dissolved in a 3:1 mixture of pyridine and piperidine (4.0 mL), and stirred at room temperature in the dark for 36 hours under argon. The solvent was completely removed under vacuum, and the residue was re-dissolved in a 60% aqueous acetone solution and passed slowly through a Dowex 50W2-100 resin in potassium form. The porphyrin fraction was collected, dried under vacuum, re-dissolved in a 30% aqueous acetone solution, and again passed through the ion-exchange resin. After removal of the solvent under vacuum, the tetraanionic porphyrin was recrystallized from methanol/diethyl ether, yielding 0.078 g (96% yield) of the title compound, m.p.>300° C. ¹H-NMR (CD₃COCD₃) δ ppm: −2.48 to −1.95 (br, 4H, BH), 0.9-2.4 (br, 32H, BH), 1.59 (s, 12H, CH₃), 3.50 (s, 8H, CH₂), 7.77 (d, 8H, ArH, J=8.0 Hz), 8.11 (d, 8H, ArH, J=8.0 Hz), 8.92 (s, 8H, β-H). UV-Vis (acetone) λₘₐₓ: 422 nm (ε 479,000), 554 (13,870), 596 (6,595).

Synthesis of Compound 7:

[3-(1-methyl-o-carboranyl)methyl]bromobenzene (7): A two-necked round bottom flask containing 1-methyl-o-carborane (3.00 g, 18.99 mmol) in dry THF (150 mL) was cooled to 0° C. under argon. n-BuLi (12.0 mL, 1.6 M in hexane) was added dropwise, and the resulting mixture was stirred at 0° C. and then cooled to −10° C. A solution of anhydrous Lil (0.350 g, 2.61 mmol) in THF (2.5 mL) was added, followed by a solution of 3-(bromomethyl)bromobenzene (5.00 g, 20.00 mmol) in THF (10 mL). After stirring at −10° C. for 15 minutes, the final reaction mixture was warmed to room temperature and stirred for 12 hours under argon. The reaction mixture was then washed with water (2×25 mL), extracted with diethyl ether (3×25 mL), and dried over Na₂SO₄. The solvent was then removed under vacuum, and the resulting crude solid was purified by column chromatography (silica gel, dichloromethane/petroleum ether 1:9) to give the title compound (4.25 g, 65.0% yield). ¹H-NMR (CDCl₃) δ ppm: 1.3-3.1 (br, 10H, BH), 2.16 (s, 3H, CH₃), 3.42 (s, 2H, CH₂), 7.13 (d, 1H, ArH, J=7.8 Hz), 7.23 (t, 1H, ArH, J=7.8 Hz), 7.33 (s,1H, ArH), 7.47 (d,1H, ArH, J=7.8 Hz).

Synthesis of Compound 8:

[3-(1-methyl-o-carboranyl)methyl]benzaldehyde (8): A solution of compound 7 (1.00 g, 3.06 mmol) in THF (25 mL) under argon was cooled to −78° C. (acetone/dry ice bath). n-BuLi (2.0 mL, 1.6 M in hexane) was added dropwise while maintaining the temperature at −78° C. The reaction mixture was stirred for 30 minutes at −78° C. before dry DMF (1.0 mL, 17.5 mmol) was slowly added. The final mixture was stirred at −78° C. for 15 minutes, and was then warmed slowly to room temperature. A 2 N HCl solution (25 mL) was added, and the reaction mixture was stirred for 2 h at room temperature. The solution was then reduced to a volume of 200 mL and extracted with dichloromethane (4×50 mL). The organic extracts were washed once with a saturated aqueous NaHCO₃ solution, and once with water, and were then dried over anhydrous Na₂SO₄. After removal of the solvent under vacuum, the oily residue was purified by column chromatography on silica gel (dichloromethane/petroleum ether 1:1), yielding the title compound (0.668 g, 79.1% yield) as a white solid. ¹H-NMR (CDCl₃) δ ppm: 1.4-3.1 (br, 10H, BH), 2.19 (s, 3H, CH₃), 3.55 (s, 2H, CH₂), 7.48 (d, 1H, ArH, J=7.8 Hz), 7.56 (t, 1H, ArH, J=7.8 Hz), 7.70 (s, 1H, ArH) 7.85 (d, 1H, ArH, J=7.8 Hz), 10.04 (s, 1H, CHO).

Synthesis of Compound 9:

meso-tetra[3-(1-methyl-o-carboranyl)methylphenyl]porphyrin (9): A solution of aldehyde 8 (0.660 g, 2.39 mmol) and freshly distilled pyrrole (0.18 mL, 2.59 mmol) in dry dichloromethane (240 mL) was purged with argon for 45 minutes. TFA (0.15 mL, 1.89 mmol) was added to the solution, and the final mixture was stirred at room temperature under argon for 18 hours. After addition of p-chloranil (0.440 g, 1.77 mmol), the final reaction mixture was stirred at room temperature for 3 hours. The organic solution was washed once with a saturated aqueous NaHCO₃ solution, and once with water, and were then dried over anhydrous Na₂SO₄. The residue obtained after removal of the solvent under vacuum was purified by column chromatography (dichloromethane/petroleum ether 1:1), and the porphyrin fraction was collected and recrystallized from dichloromethane/methanol, yielding 0.252 g (33% yield) of the title compound as purple crystals, m.p.>300° C.; MS (MALDI) m/e 1296.0 (M⁺); ¹H-NMR (CDCl₃) δ ppm: −2.84 (br, 2H, NH), 1.5-3.0 (br, 40H, BH), 2.20 (s, 12H, CH₃), 3.74 (s, 8H, CH₂), 7.62 (d, 4H, ArH), 7.74 (d, 4H, ArH), 8.05 (d, 4H, ArH), 8.18 (d, 4H, ArH), 8.84 (s, 8H, β-H). UV-Vis (CHCl₃) λₘₐₓ: 419 nm, 516, 548, 590, 646.

Synthesis of Compound 10:

meso-tetra[3-(1-methyl-nido-carboranyl)methylphenyl]porphyrin tetrapotassium salt (10): Porphyrin 9 (0.049 g, 0.0378 mmol) was dissolved in a 3:1 mixture of pyridine and piperidine (4.0 mL), and stirred at room temperature in the dark for 36 hours under argon. The solvent was completely removed under vacuum, the residue re-dissolved in a 60% aqueous acetone solution, and was then passed slowly through a Doywex 50W2-100 resin in potassium form. The porphyrin fraction was collected, dried under vacuum, re-dissolved in a 30% aqueous acetone solution, and was again passed through the ion-exchange resin. After removal of the solvent under vacuum, the tetraanionic porphyrin was recrystallized from methanol/diethyl ether, yielding 0.050 g (94% yield) of the title compound, m.p.>300° C. UV-Vis (acetone) λₘₐₓ: 431 nm, 511, 546, 590, 647.

Synthesis of Compound 13:

4-Ethynylbenzaldehyde (13): To a solution of 4-bromobenzaldehyde (10.00 g, 54.08 mmol) and triphenylphosphine (0.500 g, 1.91 mmol) in anhydrous triethylamine (80 mL) under argon were added ethynyltrimethylsilane (6.00 g, 61.09 mmol) and palladium (II) acetate (0.100 g, 0.445 mmol). The final mixture was heated to reflux for 2 hours, and was then cooled to room temperature and filtered. The filtrate was concentrated under vacuum to a thick oil, which was purified by column chromatography (dichloromethane/petroleum ether 1:4) and recrystallized from cold cyclohexane to give 10.5 g (96.1% yield) of 4-(trimethylsilylethynyl)benzaldehyde [MS m/e 187.2 (M⁺); ¹H-NMR (CDCl₃) δ ppm: 0.27 (s, 9H, SiMe₃), 7.60 (d, 2H, ArH, J=8.1 Hz), 7.82 (d, 2H, ArH, J=8.1 Hz), 10.00 (s, 1H, CHO)]. This compound (8.00 g, 39.59 mmol) was treated with K₂CO₃ (0.500 g) in methanol (50 mL) at 25° C. for 2 hours under Argon. The solvent was removed under vacuum, and the residue was dissolved in dichloromethane (100 mL). This solution was washed once with a saturated aqueous NaHCO₃ solution, and once with water, was then dried over anhydrous Na₂SO₄, and the solvent was evaporated under vacuum. The yellow residue was purified by column chromatography using dichloromethane/petroleum ether 1:4, and then recrystallized from cold cyclohexane to give 4.40 g (85.5% yield) of the title compound; MS (El) m/e 130.0; (M⁺). ¹H-NMR (CDCl₃) δ ppm: 3.30 (s, 1H, CH), 7.64 (d, 2H, ArH, J=8.1 Hz), 7.84 (d, 2H, ArH, J=8.1 Hz), 10.02 (s, 1H, CHO).

Synthesis of Compound 14:

4-(o-carboranyl)benzaldehyde (14): BF₃.OEt₂ (0.654 g, 4.62 mmol) was added under argon to a 0° C. solution of 4-ethynylbenzaldehyde (13) (6.00 g, 46.15 mmol) and 1,2-ethanedithiol (5.00 g, 53.09 mmol). The mixture was stirred at room temperature under argon for 15 minutes. The reaction mixture was then washed once with a 10% aqueous NaOH solution, and once with a saturated aqueous NaCl solution, before being dried over anhydrous Na₂SO₄. The solvent was then evaporated under vacuum. Purification of the resulting residue by column chromatography (dichloromethane/petroleum ether 1:4) gave p-ethynylbenzyl(1,3-dithiane) (7.5 g, 79% yield) as a yellow solid [MS (El) m/e 206.0 (M⁺); ¹H-NMR (CDCl₃) d ppm: 3.07 (s, 1H, CH), 3.38 and 3.51 (m, 2H each, CH₂CH₂), 5.61 (s, 1H, SCH), 7.42 (d, 2H, ArH, J=8.1 Hz), 7.47 (d, 2H, ArH, J=8.1 Hz)]. Decaborane (3.00 g, 24.59 mmol), ethyl sulfide (5.00 g, 55.44 mmol), and dry toluene (50 mL) were combined in a Schlenk tube equipped with a stir bar. This solution was heated to 40° C. for 3 hours and to 60° C. for 2 hours, and was then allowed to, cool to room temperature. To this mixture was added a solution of p-ethynylbenzyl(1,3-dithiane) (5.00 g, 24.26 mmol) in dry toluene (10 mL). The final reaction mixture was slowly warmed to 80° C., and was held at this temperature and stirred for3 days. After then cooling to room temperature, the mixture was concentrated under vacuum and the resulting oil was dissolved in methanol (250 mL) and heated to reflux until liberation of hydrogen ceased (approximately 60 minutes). At room temperature a 50% aqueous HCl solution (2 to 3 mL) was cautiously added, and the mixture was again heated to reflux until hydrogen evolution was complete (approximately 30 minutes). After cooling to room temperature, the reaction mixture was diluted with ethanol, and excess ethyl sulfide was removed by ethanol-ethyl sulfide co-distillation. The remaining residue was concentrated under vacuum. To a solution of the resulting residue in benzene (100 mL) at 5° C. was added 100 mL of a cold 10% aqueous NaOH solution, and the final mixture was stirred vigorously for 15 minutes. The organic layer was separated, washed with water (3×25 mL), and dried over anhydrous Na₂SO₄. After evaporation of the solvent, the residue obtained was purified by column chromatography (dichloromethane/petroleum ether 1:4), yielding 5.25 g (66.8% yield) of p-(o-carboranyl)benzyl(1,3-dithiane) [MS (El) m/e 324.1 (M⁺); ¹H-NMR (CDCl₃) δ ppm: 1.6-3.3 (br, 10H, BH), 3.36 and 3.47 (m, 2H each, CH₂CH₂), 3.91 (br s, 1H, o-carborane-CH), 5.56 (s, 1H, SCH), 7.40 (d, 2H, ArH, J=8.1 Hz), 7.46 (d, 2H, ArH, J=8.1 Hz)]. To a solution of this compound (5.00 g, 15.43 mmol) in 5% aqueous THF (25 mL) under argon was added dropwise a solution of HgClO₄ (12.50 g, 31.29 mmol) in THF (15 mL). The final mixture was stirred at room temperature for 15 minutes before being filtered, and the precipitate was washed 3 times with 25 mL diethyl ether. The filtrate was washed with a saturated aqueous Na₂CO₃ solution (3×25 mL) and with water (2×25 mL), before being dried over anhydrous Na₂SO₄. The residue remaining after evaporation of the solvent was purified by column chromatography (dichloromethane/petroleum ether 1:4) to give the title compound (3.27 g, 85.6% yield); MS (El) m/e 248.2 (M⁺); ¹H-NMR (CDCl₃) δ ppm: 1.60-3.2 (br, 10H, BH), 4.03 (br s, 1H, o-carborane-CH), 7.65 (d, 2H, ArH, J=8.4 Hz), 7.86 (d, 2H, ArH, J=8.4 Hz), 10.04 (s, 1H, CHO).

Synthesis of Compound 15:

meso-tetra[4-(o-carboranyl)phenyl]porphyrin (15): A solution of aldehyde 14 (1.05 g, 4.23 mmol) and freshly distilled pyrrole (0.30 mL, 4.32 mmol) in dry dichloromethane (430 mL) was purged with argon for 30 minutes. TFA (0.20 mL, 2.52 mmol) was added to the solution, and the final mixture was stirred at room temperature under argon for 24 hours. After addition of p-chloranil (0.780 g, 3.14 mmol) the final reaction mixture was stirred at room temperature for 3 hours. The solution was concentrated under vacuum to 300 mL, then washed once with a saturated aqueous NaHCO₃ solution, and once with water before being dried over anhydrous Na₂SO₄. After evaporation of the solvent under vacuum, the resulting residue was purified by column chromatography (dichloromethane/petroleum ether 1:2), and the fastest-running porphyrin fraction was collected and recrystallized from dichloromethane/methanol, yielding 0.220 g (17.7% yield) of the title compound as purple crystals, m.p.>300° C.; MS (MALDI) m/e 1184.5 (M+1); ¹H-NMR (CDCl₃) δ ppm: −2.89 (br, 2H, NH), 1.7-3.5 (br, 40H, BH), 4.28 (br s, 4H, o-carborane-CH), 7.89 (d, 8H, ArH, J=8.0 Hz), 8.17 (d, 8H, ArH, J=8.0 Hz), 8.78 (s, 8H, β-H). ¹H-NMR (d-TFA/CDCl₃) δ ppm: −0.97 (br, 4H, NH), 1.8-3.4 (br, 40H, BH), 4.31 (br s, 4H, o-carborane-CH), 8.13 (d, 8H, J=8.0 Hz), 8.51 (d, 8H, J=8.0 Hz), 8.68 (s, 8H, β-H). UV-Vis (CHCl₃) λₘₐₓ: 418 nm (ε 464,700), 514 (17,165), 550 (8,300), 590 (5,635), 646 (4,035).

Synthesis of Compound 16:

meso-tetra[4-(nido-carboranyl)phenyl]porphyrin tetrapotassium salt (16): Porphyrin 15 (0.0500 g, 0.0423 mmol) was dissolved in a 3:1 mixture of pyridine and piperidine (4.0 mL), and stirred at room temperature in the dark for 36 hours under argon. The solvent was completely removed under vacuum, the residue was re-dissolved in a 60% aqueous acetone solution, and was passed slowly through a Dowex 50W2-100 resin in potassium form. The porphyrin fraction was collected, dried under vacuum, re-dissolved in a 30% aqueous acetone solution, and again passed through the ion-exchange resin., After removal of the solvent under vacuum, the tetraanionic porphyrin was recrystallized from methanol/diethyl ether, yielding 0.0494 g (90.2% yield) of the title compound, m.p.>300° C. ¹H-NMR (CD₃COCD₃) δ ppm: −2.78 (s, 2H, NH), −2.45 to −1.90 (br, 4H, BH), 0.8-2.4 (br, 32H, BH), 2.57 (br s, 4H, nido-carborane-CH), 7.66 (d, 8H, ArH, J=8.0 Hz), 7.97 (d, 8H, ArH, J=8.0 Hz), 8.87 (s, 8H, β-H). UV-Vis (acetone) λₘₐₓ: 420 nm (ε 302,900), 516 (11,560), 554 (10,580), 594 (3,335), 650 (4,875).

Synthesis of Compound 17:

Zn(II)-meso-tetra[4-(o-carboranyl)phenyl]porphyrin (17): To a solution of porphyrin 15 (0.085 g, 0.072 mmol) in dichloromethane (50 mL), THF (4.0 mL), and pyridine (0.5 mL) was added ZnCl₂.2H₂O (0.30 g), and the final mixture was stirred at room temperature under argon overnight. The mixture was then washed once with water, dried over anhydrous Na₂SO₄, and the solvent was evaporated under vacuum. The residue was purified by column chromatography (dichloromethane/cyclohexane 1:2), and the pink-colored fraction was collected and recrystallized from dichloromethane/methanol to give 0.085 g (94.7% yield) of the title compound as purple crystals, m.p.>300° C.; MS (MALDI) m/e 1246.7 (M⁺); ¹H-NMR (CDCl₃) δ ppm: 1.6-3.6 (br, 40H, BH), 4.30 (br s, 4H, o-carborane-CH), 7.91 (br s, 8H, ArH), 8.17 (br s, 8H, ArH), 8.88 (br s, 8H, β-H). UV-Vis (CH₂Cl₂) λₘₐₓ: 424 nm (ε 607,400), 554 (22,566), 594 (6,781).

Synthesis of Compound 18:

Zn(II)-meso-tetra[4-(nido-carboranyl)phenyl]porphyrin tetrapotassium salt (18): The Zn(II) complex 17 (0.0600 g, 0.0481 mmol) was dissolved in a 3:1 mixture of pyridine and piperidine (4.0 mL), and stirred at room temperature in the dark for 36 hours under argon. The solvent was completely removed under vacuum, and the residue was re-dissolved in a 60% aqueous acetone solution and passed slowly through a Dowex 50W2-100 resin in potassium form. The porphyrin fraction was collected, dried under vacuum, re-dissolved in a 30% aqueous acetone solution and again passed through the ion-exchange,resin. After removal of the solvent under vacuum, the tetraanionic porphyrin was recrystallized from methanol/diethyl ether, yielding 0.0615 g (94.0% yield) of the title compound; ¹H-NMR (CD₃COCD₃) δ ppm: −2.54 to −1.78 (br, 4H, BH), 0.6-2.2 (br, 32H, BH), 2.58 (br s, 4H, nido-carborane-CH), 7.64 (d, 8H, ArH, J=8.1 Hz), 7.95 (d, 8H, ArH, J=8.1 Hz), 8.88 (s, 8H, β-H). UV-Vis (acetone) λₘₐₓ: 426 nm (ε 432,000), 558 (14,380), 598 (9,513).

Synthesis of Compound 19:

3-Ethynylbenzaldehyde (19): To a solution of 3-bromobenzaldehyde (10.00 g, 54.08 mmol) and triphenylphosphine (0.500 g, 1.91 mmol) in anhydrous triethylamine (100 mL) under argon were added ethynyltrimethylsilane (6.00 g, 61.09 mmol) and palladium (II) acetate (0.100 g, 0.445 mmol). The final mixture was heated to reflux for 2 hours, and was then cooled to room temperature and filtered. The filtrate was concentrated under vacuum to a thick oil, which was purified by column chromatography (dichloromethane/petroleum ether 1:4) to give 8.52 g (78.0% yield) of 3-(trimethylsilylethynyl)benzaldehyde [¹H-NMR (CDCl₃) δ ppm: 0.26 (s, 9H, SiMe₃), 7.47 (t, 1H, ArH, J=7.5 Hz), 7.70 (d, 1H, ArH, J=7.5 Hz), 7.82 (d, 1H, ArH, J=7.5 Hz), 7.96 (s, 1H ArH), 9.98 (s, 1H, CHO)]. This compound (5.00 g, 24.74 mmol) was treated with K₂CO₃ (0.500 g) in methanol (50 mL) at 25° C. for 2 hours under argon. The solvent was removed under vacuum, and the residue was dissolved in dichloromethane (100 mL). This solution was washed once with a saturated aqueous NaHCO₃ solution, and once with water, before being dried over anhydrous Na₂SO₄ and the solvent evaporated under vacuum. The yellow residue was purified by column chromatography using dichloromethane/petroleum ether 1:4 for elution, and recrystallized from cyclohexane to give 2.80 g (87.1% yield) of the title compound; MS (El) m/e 130.0; (M⁺). ¹H-NMR (CDCl₃) δ ppm: 3.20 (s, 1H, CH), 7.51 (t, 1H, ArH, J=7.8 Hz), 7.74 (d, 1H, ArH, J=7.8 Hz), 7.87 (d, 1H, ArH, J=7.8 Hz), 7.99 (s, 1H, ArH), 10.02 (s, 1H, CHO).

Synthesis of Compound 20:

3-(o-carboranyl)benzaldehyde (20): BF₃.OEt₂ (0.11 g, 0.77 mmol) was added at 0° C. under argon to a solution of 3-ethynylbenzaldehyde (19) (1.00 g, 7.69 mmol) and 1,2-ethanedithiol (0.73 g, 7.75 mmol). This mixture was stirred at room temperature under argon for 15 minutes. The reaction mixture was then washed once with a 10% aqueous NaOH solution, once with an aqueous saturated NaHCO₃ solution, and once with water before it was dried over anhydrous Na₂SO₄ and the solvent was evaporated under vacuum. Purification of the resulting residue by column chromatography (dichloromethane/petroleum ether 1:4) gave 1.28 g (80.7% yield) of m-ethynylbenzyl(1,3-dithiane) [MS (El) m/e 206.0 (M⁺); ¹H-NMR (CDCl₃) d ppm: 3.07 (s, 1H, CH), 3.37 and 3.50 (m, 2H each, CH₂CH₂), 5.59 (s, 1H, SCH), 7.27 (t, 1H, ArH, J=7.8 Hz), 7.38 (d, 1H, ArH, J=7.8 Hz), 7.50 (d, 1H, ArH, J=7.8 Hz), 7.66 (s, 1H, ArH)]. Decaborane (0.0500 g, 4.10 mmol), ethyl sulfide (0.750 g, 8.32 mmol) and dry toluene (25 mL) were combined in a Schlenk tube equipped with a stir bar. This solution was heated to 40° C. for 3 hours and to 60° C. for 2 hours, and was then allowed to cool to room temperature. To this mixture was added a solution of m-ethynylbenzyl(1,3-dithiane) (0.800 g, 3.88 mmol) in dry toluene (5 mL), and the final reaction mixture was slowly warmed to 80° C., and was stirred at this temperature for 3 days. After cooling to room temperature, the mixture was concentrated under vacuum and the resulting oil was dissolved in methanol (100 mL) and heated to reflux until liberation of hydrogen subsisted (approximately 60 minutes). At room temperature a 50% aqueous HCl solution (1.0 mL) was cautiously added, and the mixture was again heated to reflux until hydrogen evolution was complete (approximately 30 minutes). After cooling to room temperature, the reaction mixture was diluted with ethanol, and the excess ethyl sulfide was removed by ethanol-ethyl sulfide co-distillation. The remaining residue was concentrated under vacuum. To a solution of the resulting residue in benzene (50 mL) at 5° C. was added 100 mL of a cold 10% aqueous NaOH solution, and the final mixture was stirred vigorously for 15 minutes. The organic layer was separated, washed with water (3×15 mL) and dried over anhydrous Na₂SO₄. After evaporation of the solvent, the resulting residue was purified by column chromatography (dichloromethane/petroleum ether 1:4), yielding 0.905 g (72.0% yield) of m-(o-carboranyl)benzyl(1,3-dithiane) [¹H-NMR (CDCl₃) δ ppm: 1.40-3.20 (br, 10H, BH), 3.39 and 3.49 (m, 2H each, CH₂CH₂), 3.97 (br s,1H, o-carborane-CH), 5.58 (s, 1H, SCH), 7.28 (t, 1H, ArH, J=7.8 Hz), 7.39 (d, 1H, ArH, J=7.8 Hz), 7.55 (d, 1H, ArH, J=7.8 Hz), 7.63 (s, 1H, ArH)]. To a solution of the latter compound (1.00 g, 3.09 mmol) in 5% aqueous THF (10 mL) under argon was added dropwise a solution of HgClO₄ (2.50 g, 6.26 mmol) in THF (5.0 mL). The final mixture was stirred at room temperature for 15 minutes before being filtered, and the precipitate was washed with 25 mL of diethyl ether. The filtrate was then washed with a saturated aqueous Na₂CO₃ solution (3×10 mL) and with water (2×10 mL), before being dried over anhydrous Na₂SO₄. The residue remaining after evaporation of the solvent was purified by column chromatography (dichloromethane/petroleum ether 1:4) to give the title compound (0.677 g, 88.5% yield); ¹H-NMR (CDCl₃) δ ppm: 1.5-3.3 (br, 10H, BH), 4.04 (br s, 1H, o-carborane-CH), 7.56 (t, 1H, ArH, J=7.8 Hz), 7.79 (d, 1H, ArH, J=7.8 Hz), 7.91 (d, 1H, ArH, J=7.8 Hz), 7.96 (s, 1H, ArH), 10.02 (s, 1H, CHO).

Synthesis of Compound 21:

meso-tetra[3-(o-carboranyl)phenyl]porphyrin (21): A solution of aldehyde 20 (0.702 g, 2.83 mmol) and freshly distilled pyrrole (0.200 mL, 2.88 mmol) in dry dichloromethane (285 mL) was purged with argon for 30 minutes. TFA (0.100 mL, 1.26 mmol) was added to the solution, and the final mixture was stirred at room temperature under argon for 18 hours. After addition of p-chloranil (0.522 g, 2.10 mmol) the final reaction mixture was stirred at room temperature for 3 hours. The solution was concentrated under vacuum to 200 mL, then washed once with water, once with a saturated aqueous NaHCO₃ solution, and once again with water before being dried over anhydrous Na₂SO₄. After evaporation of the solvent under vacuum, the resulting residue was purified by column chromatography (dichloromethane/petroleum ether 1:2) and the fastest-running porphyrin fraction was collected and recrystallized from dichloromethane/methanol, yielding 0.140 g (16.7% yield) of the title compound as purple crystals; MS (MALDI) m/e 1184. ¹H-NMR (CDCl₃) δ ppm: −2.88 (br, 2H, NH), 1.6-3.5 (br, 40H, BH), 4.19 (br s, 4H, o-carborane-CH), 7.78 (m, 4H, ArH), 7.94 (m, 4H, ArH), 8.27 (m, 4H, ArH), 8.33 (m, 4H, ArH), 8.80 (s, 8H, β-H).

Synthesis of Compound 22:

meso-tetra[3-(nido-carboranyl)phenyl]porphyrin tetrapotassium salt (22): Porphyrin 21 (0.010 g, 0.008 mmol) was dissolved in a 3:1 mixture of pyridine and piperidine (4.0 mL), and the mixture was stirred at room temperature in the dark for 36 hours under argon. The solvent was completely removed under vacuum, and the residue was re-dissolved in a 60% aqueous acetone solution and then passed slowly through a Dowex 50W2-100 resin in potassium form. The porphyrin fraction was collected, dried under vacuum, re-dissolved in a 30% aqueous acetone solution and again passed through the ion-exchange resin. After removal of the solvent under vacuum, the tetraanionic porphyrin was recrystallized from methanol/diethyl ether, yielding 0.0108 g (98.1% yield) of the title compound, m.p.>300° C. ¹H-NMR (CD₃COCD₃) δ ppm −2.70 (s, 2H, NH), −2.40 to −1.90 (br, 4H, BH), 0.8-2.3 (br, 32H, BH), 2.48 (br s, 4H, nido-carborane-CH), 7.49 (m, 4H, ArH), 7.65 (m, 4H, ArH), 7.84 (m, 4H, ArH), 8.15 (m, 4H, ArH), 8.88 (s, 8H, β-H). UV-Vis (acetone) λₘₐₓ: 416 nm (ε 326,300), 512 (14,300), 547 (8,000), 590 (4,300), 646 (4,300).

Synthesis of Compound 25:

Bis-(3,5-bromomethyl)bromobenzene (25): To a refluxing solution of 3,5-dimethylbromobenzene (4.63 g) in dry CCl₄ (300 mL) under argon were added NBS (9.79 g) and benzoyl peroxide (0.80 g) in portions over a one hour period. The final reaction mixture was refluxed with stirring under argon for 16 hours. After cooling to room temperature, the reaction mixture was filtered, and the filtrate was washed once with a saturated aqueous NaHCO₃ solution and once with water. The organic solution was dried over anhydrous Na₂SO₄, and the solvent was evaporated under vacuum. The resulting residue was purified by column chromatography using dichloromethane/petroleum ether 1:9, and the main product was collected and recrystallized from n-hexane, yielding 2.83 g (33% yield) of the title compound; ¹H-NMR (CDCl₃) δ ppm: 4.40 (s, 2H, CH2), 7.34 (s, 1H), 7.47 (s, 2H).

Synthesis of Compound 26:

Bis[3,5-(1-methyl-o-carboranyl)methyl]bromobenzene (26): n-BuLi (5.2 mL, 1.6 M in hexane) was added dropwise to a solution of methyl-o-carborane (1.39 g, 8.80 mmol) in dry THF (80 mL), at a temperature between −5° and 0° C. under argon. The mixture was stirred at this temperature range for 90 minutes, and then cooled to −15° to −20° C. (ice/salt bath). A solution of Lil (0.166 g, 1.27 mmol) in dry THF (15 mL) and Compound 25 (1.372 g, 4.00 mmol) was added, and the final reaction mixture was allowed to warm to room temperature and was stirred for 16 hours. After the reaction was quenched with water, the resulting mixture was extracted with diethyl ether. The organic extracts were washed once with water and once with brine, were dried over anhydrous Na₂SO₄, and the solvent was removed under vacuum. The crude product was purified by column chromatography using dichloromethane/petroleum ether 2:8 for elution, and the main product was collected and recrystallized from n-hexane to give 1.26 g (63% yield) of the title compound; MS m/e 497.3; ¹H-NMR (CDCl₃) δ ppm: 1.4-3.0 (br, 20H, BH), 2.17 (s, 6H, CH₃), 3.43 (s, 4H, CH₂), 6.96 (s, 1H), 7.31 (s, 2H).

Synthesis of Compound 27:

Bis[3,5-(1-methyl-o-carboranyl)methyl]benzaldehyde (27): A solution of compound 26 (0.994 g) in THF (20 mL) under argon was cooled to −78° C. n-BuLi (1.4 mL, 1.6 M in hexane) was added dropwise via syringe. After the reaction mixture was stirred for 30 minutes at −78° C., dry DMF (0.77 mL) was slowly added. The resulting yellow mixture was stirred at −78° C. for 30 minutes, and was then warmed to 0° C. and stirred at this temperature for one hour. A 5% aqueous HCl solution was added until the pH of the reaction mixture was between 2 and 3, and the final mixture was then stirred at room temperature. The aqueous layer was extracted 4 times with diethyl ether, the organic fraction was dried over anhydrous MgSO₄, and the solvent was evaporated under vacuum. Purification by column chromatography (dichloromethane/petroleum ether 2:3), afforded the title compound (0.632 g) in 70.9% yield; MS m/e 446.4; ¹H-NMR (CDCl₃) δ ppm: 1.5-3.0 (br, 20H, BH), 2.20 (s, 6H, CH₃), 3.55 (s, 4H, CH₂), 7.30 (d, 1H, J=1.6 Hz), 7.67 (d, 2H, J=1.6 Hz), 10.03 (s, 1H, CHO).

Synthesis of Compound 28:

meso-tetra[bis-3,5-(1-methyl-o-carboranyl)methylphenyl]porphyrin (28): A solution of aldehyde 27 (0.243 g, 0.54 mmol) and freshly distilled pyrrole (0.050 mL g, 0.72 mmol) in dry dichloromethane (55 mL) was purged with argon for 15 minutes. TFA was added, and the final solution was stirred at room temperature overnight (until the starting aldehyde had completely disappeared, and 2 new spots had formed as assayed by TLC). After oxidation with p-chloranil (0.102 g, 0.41 mmol) for 6 hours at room temperature, the final reaction mixture was washed once with a saturated aqueous NaHCO₃ solution and once with water before being dried over anhydrous Na₂SO₄. The residue obtained after removal of the solvent was purified by column chromatography using dichloromethane/petroleum ether 1:2 for elution. The porphyrin fraction obtained was recrystallized from dichloromethane/methanol, to give 0.30 g (12% yield) of the title compound; MS m/e 1977.3 ¹H-NMR (d-TFA/CDCl₃) δ ppm: −0.80 (br, NH), 1.5-3.1 (br, 80H, BH), 2.31 (s, 24H, CH₃), 3.91 (s, 16H, CH₂), 7.72 (s, 4H), 8.33 (s, 8H), 8.74 (s, 8H, β-H).

Synthesis of Compound 30:

5,15-bis[bis-3,5-(1-methyl-o-carboranyl)methylphenyl]porphyrin (30): A solution of aldehyde 27 (0.446 g) and dipyrromethane 29 (0.146 g) in dry dichloromethane (100 mL) was purged with argon for 15 minutes and cooled to 0° C. TFA was added to the solution, and the final mixture was stirred at 0° C. for 2 hours and then at room temperature overnight. After oxidation with p-chloranil (0.277 g, 1.13 mmol) for 6 hours at room temperature, the final reaction mixture was washed once with a saturated aqueous NaHCO₃ solution, once with water, and once with brine before being dried over anhydrous MgSO₄. The residue obtained after removal of the solvent was purified by column chromatography (alumina) using dichloromethane for elution. The porphyrin fraction obtained was recrystallized from acetone to give 33.6% yield (0.192 g) of the title compound; MS m/e 1144.0; ¹H-NMR (d-TFA/CDCl₃) δ ppm: −1.92 (br, NH), 1.4-3.2 (br, 40H, BH), 2.30 (s, 12H, CH₃), 3.89 (s, 8H, CH₂), 7.71 (s, 2H), 8.34 (s, 4H) 9.03 (d, 4H, β-H, J=4.5 Hz), 9.61 (d, 4H, β-H, J=4.5 Hz), 10.98 (s, 2H, meso-H).

Synthesis of Compound 31:

5,15-bis[bis-3,5-(1-methyl-nido-carboranyl)methylphenyl]porphyrin tetrapotassium salt (31): Porphyrin 30 (0.100 g) was dissolved in a 3:1 mixture of pyridine and piperidine (4.0 mL), and stirred at room temperature in the dark for 36 hours under argon. The solvent was completely removed under vacuum, the residue was re-dissolved in a 60% aqueous acetone solution and passed slowly through a Dowex 50W2-100 resin in potassium form. The porphyrin fraction was collected, dried under vacuum, re-dissolved in a 30% acetone aqueous solution and again passed through the ion-exchange resin. After removal of the solvent under vacuum, the tetraanionic porphyrin was recrystallized from methanol/diethyl ether, yielding 0.102 g (92.8% yield) of the title compound. ¹H-NMR (CD₃COCD₃) δ ppm: −2.84 (s, 2H, NH), −2.45 to −1.85 (br, 4H, BH), 0.9-2.4 (br, 32H, BH), 1.66 (s, 12H, CH₃), 3.52 (s, 8H, CH₂), 7.67 (s, 1H, ArH), 7.74 (s, 1H, ArH), 8.37 (s, 4H, ArH), 9.53 (dd, 4H, β-H), 9.61 (dd, 4H, β-H), 10.58 (s, 2H, meso-H). UV-Vis (acetone) λₘₐₓ: 406 nm (ε 312,600), 502 (13,400), 536 (7,800), 576 (6,100), 630 (3,100).

Synthesis of Compound 32:

Zn(II)-5,15-bis[bis-3,5-(1-methyl-o-carboranyl)methylphenyl]porphyrin (32): To a solution of porphyrin 30 (0.065 g, 0.057 mmol) in dichloromethane (100 mL) and THF (10 mL) was added ZnCl₂.2H₂O (0.031 g, 0.288 mmol), and the final mixture was stirred at room temperature under argon overnight. The mixture was then washed once with water, dried over anhydrous Na₂SO₄, and the solvent was evaporated under vacuum. The residue was purified by column chromatography (dichloromethane/cyclohexane 2:1), and the pink-colored fraction was collected and recrystallized from dichloromethane/methanol to give 0.061 g (89% yield) of the title compound. ¹H-NMR (CDCl₃) δ ppm: 1.4-3.0 (br, 40H, BH), 2.11 (s, 12H, CH₃),3.64 (s, 8H, CH₂),7.33 (s, 2H, ArH), 7.98 (s, 4H, ArH), 8.98 (d, 4H, β-H, J=4.5 Hz), 9.41 (d, 4H, β-H, J=4.5 Hz), 10.25 (s, 2H, meso-H).

Synthesis of Compound 33:

Zn(II)-5,15-bis[bis-3,5-(1-methyl-nido-carboranyl)methylphenyl]porphyrin tetrapotassium salt (33): The Zn(II) complex 32 (0.050 g) was dissolved in a 3:1 mixture of pyridine and piperidine (4.0 mL), and was stirred at room temperature in the dark for 36 hours under argon. The solvent was completely removed under vacuum, the residue was re-dissolved in a 60% aqueous acetone solution, and was passed slowly through a Dowex 50W2-100 resin in potassium form. The porphyrin fraction was collected, dried under vacuum, re-dissolved in a 30% aqueous acetone solution and again passed through the ion-exchange resin. After removal of the solvent under vacuum, the tetraanionic porphyrin was recrystallized from methanol/diethyl ether, yielding 0.052 g (94.8% yield) of the title compound; ¹H-NMR (CD₃COCD₃) δ ppm: −2.45 to −1.85 (br, 4H, BH), 0.9-2.4 (br, 32H, BH), 1.65 (s, 12H, CH₃), 3.50 (s, 8H, CH₂), 7.66 (s, 1H, ArH), 7.73 (s, 1H, ArH) 8.28 (s, 4H, ArH), 9.41 (dd, 4H, β-H), 9.47 (dd, 4H, β-H), 10.33 (s, 2H, meso-H). UV-Vis (acetone) λₘₐₓ: 412 nm (ε 263,100), 496 (1,400), 542 (9,900), 580 (1,070), MS (MALDI) m/e 1319.4.

In Vitro and In Vivo Analysis of Boronated Porphyrins

Biological Studies

In vitro studies using rat 9L gliosarcoma cells, mouse B16 melanoma cells, hamster V79 fibroblast cells, and human U-373MG glioblastoma cells have been conducted. We found that all compounds studied had very low dark cytotoxicities, were readily taken up and retained by cells, and were localized in specific cell organelles, primarily those in close proximity to the cell nucleus.

Our biological studies to date have indicated that these compounds have very low in vivo toxicities. So far we have determined maximum tolerated doses for compounds 4, 6, 16, 31, and 33 using healthy female Balb/c mice. For all compounds tested, we found that the maximum tolerated dose exceeded 300 mg per kg of body weight.

Cytotoxicity/Phototoxicity Assays

Human glioblastoma U373 MG, hamster V79 fibroblast, and mouse melanoma B16 cells were obtained from the American Type Culture Collection. Rat gliosarcoma cells were kindly provided by the University of California at San Francisco Brain Tumor Research Group. All cells were maintained in log phase monolayer cultures with RPMI 1640 supplemented with 10% fetal bovine serum and 2 mM glutamine.

Cells were seeded in 96-well culture plates, and were allowed to settle and attach for 24-48 hours. Triplicate wells were then exposed to twofold serial dilutions of test compounds at concentrations up to 250 μM. Compounds 4, 6, 10, and 12 in crystalline form were carefully weighed and dissolved in 100% DMSO to prepare stock solutions. Subsequent dilutions were performed directly in the culture medium just before the medium was administered to cells. After short-term (2 hour) or long-term (24 hour) exposure, cells were washed, and the wells were refilled with fresh culture medium. For dark toxicity trials, cells were allowed to proliferate for an additional 48-72 hours. For phototoxicity trials, washed cells were irradiated for 10 minutes with broad spectrum (600-700 nm) red light, and were then returned to an incubator for 48-72 hours.

Exposure to one of the compounds for 2 hours, followed by washing out the compound, did not inhibit proliferation of any of the three cell types. Exposure for 24 hours was inhibitory only at the higher concentrations (IC₅₀≧150 μM) for 9L and U-373 MG cells, but B16 viability was unaffected. Whereas the metal-free porphyrins 4 and 10 displayed nearly identical IC₅₀ values (˜150 μM) in the affected cells, their Zn(II) complexes were about 20% less toxic (IC₅₀˜180-185 μM).

We have also determined the cytotoxicity of porphyrins 4, 16, 18, and 31 in V79 hamster fibroblast cells using a clonogenic assay. For each of these porphyrins, we found CS₅₀>300 μM, and we observed no toxicity (i.e., no effect on colony survival) at concentrations up to 200 μM.

In summary, these results showed that the nido-carboranyl compounds of the present invention display low dark toxicity.

For carboranylporphyrin 4, irradiation with broad spectrum light caused cytotoxicity at 2-hour (IC₅₀˜50 μM), and 24-hour exposures (IC₅₀˜1.5 μM). Similar to the dark toxicity experiments, the corresponding Zn(II) complex 6 was also found to be about 20-fold less phototoxic. Similar to the dark toxicity results, the B16 cells were found to be more resistant than the other two cell types.

Cellular Uptake and Retention

The concentration-dependent uptake of compounds 4, 6, 10, and 12 was investigated in cells exposed to 1, 5, and 10 μM concentrations of the porphyrins for 24 hours. The concentration of intracellular-bound porphyrin was determined by chemical extraction of washed cell monolayers, followed by spectrophotometric or ICP-MS determinations, or both. The uptake values for 9L and U-373MG cells were very similar, and exceeded that for B16 cells. The porphyrin accumulation was invariably observed to increase with increasing concentration of the compound. The uptake of nido-carboranylporphyrins 4 and 10 was approximately four times greater than that of the corresponding Zn(II) complexes.

The uptake of carboranylporphyrins by log phase cells was also shown to be time-dependent. Cells exposed to 5 μM concentrations of the compounds contained increasing amounts of extractable porphyrin over a 24-hour uptake period. While 9L and U-373MG cells had similar uptake levels, B16 cell cultures consistently accumulated 60-70% less of the compounds per cell. Cell-bound porphyrin that could not be removed by rinsing the cells with Hanks balanced salt solution was detectable as early as one hour after the compound was introduced into the cell culture. In experiments using 9L cells exposed to a 10 μM concentration of free-base porphyrins 4 and 10, intracellular levels>60 μg of boron per billion cells (or gram of wet tissue) were achieved following a 24-hour exposure to the compound.

Intracellular Localization

Confocal fluorescence microscopy was used to examine the intracellular location of nido-carboranylporphyrin 4 in live cells. Rat 9L tumor cells and human normal keratinocyte line HaCaT were used in these studies. HaCaT cells were included in these particular experiments because they adhere to and spread readily on glass cover slips, thus facilitating imaging. Cells exposed to a 2 μM concentration of porphyrin 4 for either 6 or 24-hours were examined at a magnification of 200× for intracellular fluorescence using excitation/emission wavelengths optimized for this type of compound. The 9L and HaCaT cells showed a similar intracellularfluorescence pattern. In both cases, 100% of the cells were labeled; the cells exposed to the compound for 24 hours were slightly brighter than the cells that had been exposed for 6 hours. The punctuate fluorescence was predominantly perinuclear, with many cells having an additional local area of concentration that appeared to be adjacent to the nuclear membrane. No fluorescence signal was detectable in the plasma membrane of isolated individual cells, nor in the intercellular junctions of confluent cells.

Assay for In Vitro Damage

Following light activation the nido-carboranylporphyrins 16, 31 and 33 effectively induced DNA damage in vitro. Two different methods were used to assess the extent of DNA damage: measuring the ratio of super-coiled DNA to nicked DNA, and the alkaline Comet assay using human leukemia K562 cells. Significant photo-induced DNA damage was observed for porphyrins 16, 31 and 33 with both assays, as compared to light-only and porphyrin-only control experiments.

In the first test to determine the ability of a porphyrin to induce DNA photodamage, we used a supercoiled DNA substrate that migrates rapidly as a single band in agarose gels, but migrates much more slowly if the DNA sustains as little as a single phosphodiester break or nick. Using this technique, we examined the ability of porphyrins 16, 31 and 33 to nick the supercoiled DNA substrate φX174. The porphyrin—DNA mixtures were shielded from light until just prior to the nicking assay, at which time half of each sample remained in the dark, while the other half was exposed to yellow light from a 100 W bulb at a distance of 30 cm for 30 minutes, at room temperature. The samples were then incubated for 30 min at room temperature in the dark, and the products of the reaction were then electrophoresed on a 1% agarose gel and stained. The samples that had been exposed to light were significantly more effective (P≦0.05) in causing phosphodiester breaks in DNA. Densitometer analysis of stained gels revealed that the samples remaining in the dark produced little Form II DNA, as did the light-only controls; while the samples exposed to light produced significantly higher DNA damage.

The second test, the alkaline Comet assay, is a sensitive technique for determining the presence of single- and double-strand breaks and other DNA lesions such as baseless sites. Human K562 cells were incubated with porphyrins 16, 31, and 33, and were then either exposed to a source of yellow light from a 100 W bulb for 5 minutes at room temperature or kept in the dark. The cells were then immobilized on agarose slides, lysed, exposed to alkaline buffer, electrophoresed, and stained for visualization. Comparisons between the porphyrin-treated cells exposed to light and those kept in the dark showed that the light-exposure significantly intensified the formation of the Comet tail, indicating DNA damage. Porphyrins 16, 31, and 33 caused light-activated DNA damage that was significantly different (P<0.05) both from controls exposed to the porphyrin but held in the dark, and controls exposed to light without the porphyrin. These results demonstrated that the nido-carboranylporphyrins will cause photoactivated DNA damage.

Assay for In Vitro Virucidal Activity

Porphyrins 16, 31, and 33 have been assayed for in vitro virucidal activity against HIV-1 using the assay of A. Vzorov et al. (2002) (full citation below). The assay was conducted on two different strains of HIV-1, strain IIIB, and strain 89.6. The results are shown in the table below. The figures in the table denote the percentage of viral particles that were still infective following treatment. A figure of 0% means that essentially no infective particles remained following treatment, while a figure of 100% would mean that the treatment caused essentially no reduction in infectivity.

| | HIV-1 strain IIIB | HIV-1 strain 89.6 |
|---|---|---|
| Compound 16 | 4% | 0% |
| Compound 31 | 9% | 2% |
| Compound 33 | 0% | 4% |

These data suggest that Compounds 16, 31, and 33 are effective against various strains of HIV-1.

Miscellaneous

Compounds used in the present invention may be administered to a patient for treatment or prevention of viral infection by any suitable means, including oral, intravenous, parenteral, subcutaneous, intrapulmonary, and intranasal administration. Parenteral infusions include intramuscular, intravenous, intraarterial, or intraperitoneal administration. The compounds may also be administered transdermally, for example in the form of a slow-release subcutaneous implant, or orally in the form of capsules, powders, or granules, with or without a coating such as an enteric coating. They may also be administered by inhalation.

Pharmaceutically acceptable carrier preparations for parenteral administration include sterile, aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. The active therapeutic ingredient may be mixed with excipients that are pharmaceutically acceptable and are compatible with the active ingredient. Suitable excipients include water, saline, dextrose, glycerol and ethanol, or combinations thereof. Intravenous vehicles include fluid and nutrient replenishers, electrolyte replenishers, such as those based on Ringers dextrose, and the like. Preservatives and other additives may also be present such as, for example, antimicrobials, anti-oxidants, chelating agents, inert gases, and the like.

The form may vary depending upon the route of administration. For example, compositions for injection may be provided in the form of an ampule, each containing a unit dose amount, or in the form of a container containing multiple doses.

The compound may be formulated into therapeutic compositions as pharmaceutically acceptable salts. These salts include acid addition salts formed with inorganic acids, for example hydrochloric or phosphoric acid, or organic acids such as acetic, oxalic, ortartaric acid, and the like. Salts also include those formed from inorganic bases such as, for example, sodium, potassium, ammonium, calcium or ferric hydroxides, and organic bases such as isopropylamine, trimethylamine, histidine, procaine and the like.

A method for controlling the duration of action comprises incorporating the active compound into particles of a polymeric substance such as a polyester, peptide, hydrogel, polylactide/glycolide copolymer, or ethylenevinylacetate copolymers. Alternatively, an active compound may be encapsulated in microcapsules prepared, for example, by coacervation techniques or by interfacial polymerization, for example, by the use of hydroxymethylcellulose or gelatin-microcapsules or poly(methylmethacrylate) microcapsules, respectively, or in a colloid drug delivery system. Colloidal dispersion systems include macromolecule complexes, nanocapsules, microspheres, beads, and lipid-based systems including oil-in-water emulsions, micelles, mixed micelles, and liposomes.

Initial in vivo animal trials will be conducted in accordance with all applicable laws and regulations, following by clinical trials in humans in accordance with all applicable laws and regulations.

As used in the specification and claims, an “effective amount” of a compound is an amount, that when administered to a patient (whether as a single dose or as a time course of treatment), inhibits or prevents viral infection to a statistically significant degree as compared to control. “Statistical significance” means significance at the P<0.05 level, or such other measure of statistical significance as would be used by those of skill in the art of biomedical statistics in the context of a particular type of treatment or prophylaxis.

The complete disclosures of all references cited in this specification are hereby incorporated by reference. Also incorporated by reference are the complete disclosures of the following papers, neither of which is believed to be prior art against the present application: A. Vzorov et al., “Inactivation of human immunodeficiency virus type I by porphyrins,” Antimicrobial Agents & Chemotherapy, vol. 46, pp. 3917-3925 (2002); and A. Vzorov et al., “Prevention of HIV-1 infection by phthalocyanines,” Antiviral Research, vol. 59, pp. 99-109(2003). Also incorporated by reference are the complete disclosures of the following two published international patent applications, including particularly, but not limited to, the antiviral and virucidal assays disclosed in the applications: Compans et al., “Porphyrins with virucidal activity,” published as international patent application WO 03/057176; and Compans et al., “Phthalocyanines and porphyrazine pharmaceutical compositions,” published as international patent application WO 03/061579. Also incorporated by reference are the complete disclosures of the following two provisional applications: Vicente and Marzilli, “Treating and preventing viral infections with porphyrin-based compounds,” U.S. provisional patent application serial No. 60/426,062, filed Nov. 13, 2002; and Vicente, “Chelation of charged and uncharged molecules with porphyrin-based compounds,” U.S. provisional patent application serial No. 60/426,612, filed Nov. 15, 2002. In the event of an otherwise irreconcilable conflict, however, the present specification shall control.

We claim:

1. A method for inhibiting or preventing a viral infection in a patient, said method comprising administering to the patient an effective amount of a compound comprising a porphyrin macrocycle, and further comprising one or more carboranyl groups that are linked to the porphyrin macrocycle by carbon-carbon bonding.

2. A method as recited in claim 1, wherein the patient is a human.

3. A method as recited in claim 2, wherein the method inhibits or prevents infection by human immunodeficiency virus.

4. A method as recited in claim 1, wherein the compound has structure I: 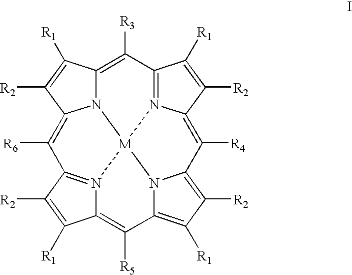
wherein M is 2H or a metal ion; R1 and R2 are each independently hydrogen, C₁ to C₄ alkyl or hydroxyalkyl; and R3, R4, R5, and R6 are each independently hydrogen, phenyl, or substituted phenyl having structure II: 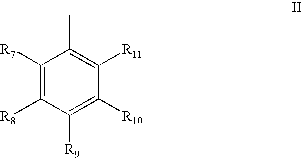
wherein R7, R8, R9, R10, and R11 are independently hydrogen or a carboranyl group, wherein such a carboranyl group is linked to the phenyl group by a carbon-carbon bond; and wherein one or two of R7, R8, R9, R10, and R11 are hydrogen, halide, hydroxide, alkoxide, sulfonate, or a substituted or unsubstituted alkyl or aryl; or such a carboranyl group; and
wherein at least one of R3, R4, R5, and R6 is a substituted phenyl having structure II and having at least one such a carboranyl group.

5. A method as recited in claim 4, wherein at least two of R3, R4, R5, and R6 are substituted phenyls having structure II and each having at least one such a carboranyl group.

6. A method as recited in claim 4, wherein each of R3, R4, R5, and R6 is a substituted phenyl having structure II and each having at least one such a carboranyl group.

7. A method as recited in claim 4, wherein at least two of R3, R4, R5, and R6 are substituted phenyls having structure II and each having at least one such a carboranyl group.

8. A method as recited in claim 1, additionally comprising the step of exposing tissue of the patient to light having a wavelength, intensity, and duration sufficient to significantly enhance the compound's inhibition or prevention of viral infection.

9. A method as recited in claim 1, wherein the compound is selected from the group consisting of Compounds 4, 6, 10, 12, 16, 18, 22, 24, 31, and 33.

10. A method as recited in claim 1, wherein the compound is Compound 16.

11. A method as recited in claim 1, wherein the compound is Compound 31.

12. A method as recited in claim 1, wherein the compound is Compound 33.

13. A method for killing or inhibiting viruses in or on a material, said method comprising treating the material with an effective amount of a compound comprising a porphyrin macrocycle, and further comprising one or more carboranyl groups that are linked to the porphyrin macrocycle by carbon-carbon bonding.

14. A method as recited in claim 13, wherein the method kills or inhibits the human immunodeficiency virus.

15. A method as recited in claim 13, wherein the compound has structure I: 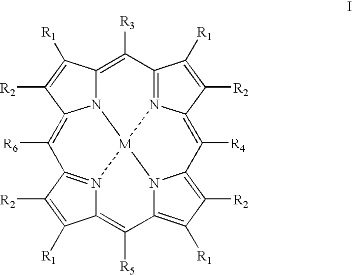
wherein M is 2H or a metal ion; R1 and R2 are each independently hydrogen, C₁ to C₄ alkyl or hydroxyalkyl; and R3, R4, R5, and R6 are each independently hydrogen, phenyl, or substituted phenyl having structure II: 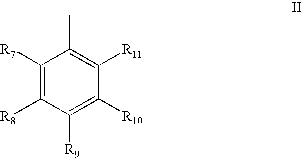
wherein R7, R8, R9, R10, and R11 are independently hydrogen or a carboranyl group, wherein such a carboranyl group is linked to the phenyl group by a carbon-carbon bond; and wherein one or two of R7, R8, R9, R10, and R11 are hydrogen, halide, hydroxide, alkoxide, sulfonate, or a substituted or unsubstituted alkyl or aryl; or such a carboranyl group; and
wherein at least one of R3, R4, R5, and R6 is a substituted phenyl having structure II and having at least one such a carboranyl group.

16. A method as recited in claim 15, wherein at least two of R3, R4, R5, and R6 are substituted phenyls having structure II and each having at least one such a carboranyl group.

17. A method as recited in claim 15, wherein each of R3, R4, R5, and R6 is a substituted phenyl having structure II and each having at least one such a carboranyl group.

18. A method as recited in claim 15, wherein at least two of R3, R4, R5, and R6 are substituted phenyls having structure II and each having at least one such a carboranyl group.

19. A method as recited in claim 13, additionally comprising the step of exposing the material to light having a wavelength, intensity, and duration sufficient to significantly enhance the compound's killing or inhibition of viruses.

20. A method as recited in claim 13, wherein the compound is selected from the group consisting of Compounds 4, 6, 10, 12, 16, 18, 22, 24, 31, and 33.

21. A method as recited in claim 13, wherein the compound is Compound 16.

22. A method as recited in claim 13, wherein the compound is Compound 31.

23. A method as recited in claim 13, wherein the compound is Compound 33.
